(19) United States
(12) Patent Application Publication
(10) Pub. No.: US 2004/0260223 A1
(43) Pub. Date: Dec. 23, 2004

(54) External fixation device

(76) Inventors: Thomas S. Roukis, Arlington Heights, IL (US); Adam S. Landsman, Chicago, IL (US)

Correspondence Address: Michael B. McMurry, 1210 Astor Street., Chicago, IL, 60610, US

(21) Appl. No.: 10/869,584

(22) Filed: Jun. 16, 2004

Related U.S. Application Data
(60) Provisional application No. 60/478,838, filed on Jun. 17, 2003.

(51) Int. Cl. A61F 5/00

(52) U.S. Cl. 602/32

(57) ABSTRACT

An external fixation device is provided to relieve pressure from the lower leg by elevating it above a support surface. The device includes a frame, a supporting member and transfixation wires that are inserted into a lower leg bone and at least one foot bone. The transfixation wires are adjustably connected to the support member which in turn is adjustably connected to the frame. The device can be adjusted for position, height, and also for limited movement of the foot.

### FIELD OF THE INVENTION

The invention relates to a medical device which can be attached to the body by wires, clamps, screws, pins, or similar devices passing through the soft tissues and in particular into or through the bones of the leg, lower leg and foot in order to prevent a limb from resting on a bed or other supporting surface.

### BACKGROUND OF THE INVENTION

In the past, medical professionals who performed delicate surgical procedures on the heel and posterior leg have dealt with excessive post-operative pressure to this region using devices such as pressure relieving posterior leg plaster/fiberglass splints; surgical gloves filled with water as a “mini-water mattress”; and fixing the lower leg within several pillows as a “pillow box”. However, convalescent patients still frequently develop decubitus ulcerations on the posterior heel region which results in a substantial medical as well as an economic burden.

A method to overcome this problem has been described by Gregory A. Buford, M.D. and Marc A. Trzeciak, D.O. in an article titled “A Novel Method for Lower-Extremity Immobilization after Free-Flap Reconstruction of Posterior Heel Defects” in the February 2003 publication of PLASTIC AND RECONSTRUCTIVE SURGERY. This method and device is designed to prevent pressure to these areas by suspending the heel over the mattress or other contact surface, minimizing or totally eliminating any contact between the heel and the mattress or other contact surface as the patient remains supine in their bed or chair. By suspending the heel, the potential for formation of bed sores or decubitus pressure ulcers of any kind is virtually eliminated. But, this device and method as described by Buford and Trzeciak has a number of disadvantages. For example, the device is multi-articulated and requires a significant number of parts to construct, which raises the cost of manufacturing as well as the time and cost of assembly. Also, this device does not allow the lower leg to be elevated completely allowing the back of the calf and knee region to come in contact with the bedding or other support surfaces. As a result, this device would not be suitable for use with the calf region where flaps can be harvested and skin grafts applied. Moreover, since this device is only intended to relieve pressure from the heel area, it can complicate recovery for any soft tissue reconstruction about the midfoot, ankle, or lower leg. In addition, the device uses pins in the 5^{th} metatarsal and 1^{st} metatarsal which limits the versatility of the device and accessibility to the foot.

### SUMMARY OF THE INVENTION

It is therefore an object of the invention to provide a device, using a minimum of components, in which wires, clamps, screws, pins or similar devices pass onto, into, or through the lower leg and foot and are attached to a frame which holds the entire lower leg, ankle, heel, and foot suspended in space, so as not to create pressure between the surfaces of the leg, ankle, heel, and foot, and a mattress or similar support surface beneath.

A further object of the invention is to allow flexibility in the fixation of the pins to the dorsal or inner aspect of the foot, which permits greater speed in application, accessibility, versatility, and limits the dissection, soft-tissue and osseous trauma, and potential number of pin-site tracts for infection, yet allows for stability.

Still another object of the invention is to allow for any soft-tissue reconstruction and relieve pressure about the midfoot, heel, ankle, and lower leg since the device can be used to off load the entire limb.

### BRIEF DESCRIPTION OF THE DRAWINGS

FIG. 1 is a front view of an external fixation device according to the invention; and

FIG. 2 is a side view of the external fixation device of FIG. 1.

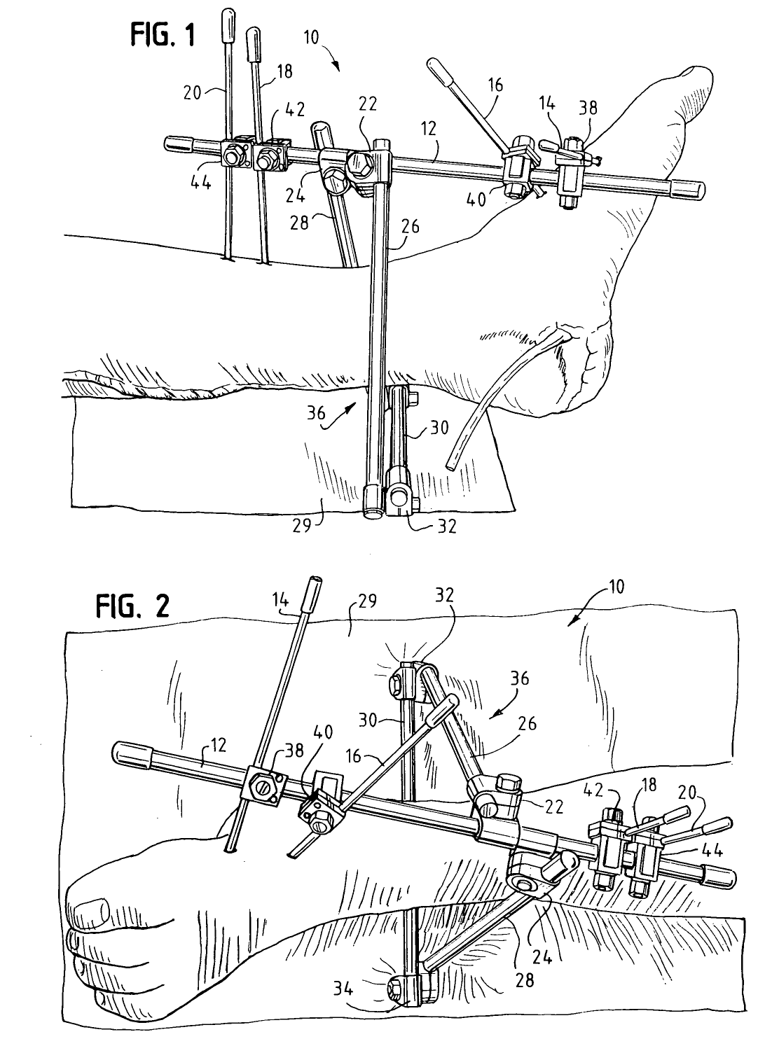

### DETAILED DESCRIPTION OF THE INVENTION

The elements of an external fixator device 10 according to the invention can be constructed from components obtained, for example from Synthes of Paoli, Pa., resulting in large external fixator system having the characteristics of simplicity, versatility, durability, and convenience. In addition to the description provided below, the disclosure of provisional patent application Ser. No. 60/478,836, filed on Jun. 17, 2003 is incorporated by reference. The preferred embodiment of the external fixator 10 includes a carbon fiber support rod 12, preferably 400-mm in length oriented along the anterior-medial aspect of the distal tibia and foot. In this example of the invention, the support rod 12 is connected to the medial face of the distal tibia and the foot using four transfixation wires 14-20 that are 5.0-mm in diameter, 50-mm in thread length, and 200-mm in length with blunted trocar points, self-tapping screws and adjustable clamps. The first two of these transfixation wires, 18 and 20, are inserted into the tibia and the remaining two transfixation wires, 14 and 16, into bones of the foot, preferably one in the first or second metatarsal and the second in the first cuneiform. Using a pair of tube-to-tube clamps 22 and 24, two additional carbon fiber frame members 26 and 28, preferably 350-mm in length are attached to the carbon fiber support rod 12 just distal to the tibial transfixation wire attachment sites and are aligned in the shape of a triangle being angled medially and laterally away from the lower leg to contact a weight bearing support surface such as a mattress 29 beneath. Finally, a carbon fiber base member 30 preferably measuring 400-mm in length is connected across the weight bearing support surface 29 to the medial and lateral angled carbon fiber frame members 26 and 28 using a pair of tube-to-tube clamps 32 and 34. Once completed, the limb remains suspended above the weight bearing support surface 29 in the upper portion of the triangular frame 36.

The shape of the supporting frame 36 can be varied to accommodate different treatment conditions. For instance, the support frame 36 can have a rounded, triangular, trapezoidal or square shape in order to either enhance or restrict side-to-side motion of the leg, as desired. As an example, a U-shaped support can be used to allow limited natural motion of the leg. Parallel supports can also be used to enhance stability. The support frame 36 and rods can be created in a variety of shapes, and from a variety of materials, including but not limited to plastic, composites, metals, or any combination thereof. There can be a wide variety of frames, which may be either adjustable or non-adjustable, in order to modify the design as necessary, to facilitate changes in position and changing of bandages.

The device 10 is based on a system of transfixation of the leg or a portion of the leg along with the foot with wires, pins, screws, or clamps either securely attaching to the bones; passing into the bones; or completely traversing the bones involved.

A preferred method of using the fixation device 10 involves the use of an appropriate diameter drill and protective tissue sleeve to first create holes in the bone. Then either the bone is tapped to the proper thread pattern of the transfixation wires 14-20 or self-tapping transfixation wires can be used. In addition, it is preferable to use an antibiotic bacteriostatic or bacteriocidal coating incorporated into the transfixation wires 14-20 to prevent bacterial growth and subsequent infection from developing around the insertion rod site, that is pin-tract infection. Once transfixated, the transfixation wires 14-20 are attached to the support rod 12 via a set of adjustable clamps 38-44. As a result, the frame 36 is effective to support the limb in space, eliminating or greatly reducing contact between the limb and the supporting surface 29. Placement of the transfixation device 36 can be done in a wide variety of positions, based on the condition of the limb, and the areas where access to the limb may be necessary. For most applications, the principle support of the limb will come from the insertion of the two pins 14 and 16 in the anterior tibia. Alternatively, the transfixation wires 14-20 can be inserted in the frontal plane through the tibia or clamped onto the outer surface of the bone. Secondary stabilization of the foot can be achieved when motion at the ankle is potentially detrimental, such as in the case of a large skin flap to the posterior heel, or an ankle procedure. Stabilization of the foot can also be performed in a variety of ways, such as with transfixation pins inserted into the calcaneous, metatarsal, or other foot bones.

The frame 36 is designed to accomplish the principal task of supporting the leg and foot in space while resting on the supporting surface or mattress 29. The frame 36 can be prefabricated in a variety of shapes, or can be assembled on a custom basis at the time of device application. Depending on the design selected, the frame can either restrict or promote rotation of the limb. In the preferred embodiment, the frame 36 is connected to the support rod 12 using the clamps 22 and 24 which can have the angle created between the frame members 26 and 28 fixed or allow the ability to move in multiple directions. A nut-and-bolt configuration allows the clamps 22 and 24 to be securely tightened with hand instrumentation customized to the size (i.e. diameter) of the nut-and-bolt system.

The device 10 is designed to rest on the mattress or other supporting surface. Several variations of the frame 36 can be used, including a design that will permit the leg to rotate from side to side, and one that will hold the leg in a single position. The device 10 can be assembled in advance or a pre-formed device can be used. Adjustments at the bedside can be made for frame height, position, and a multitude of other configuration.

We claim:

1. An external fixation apparatus for supporting a human leg above a surface comprising:
a support frame adapted for placement on the surface;
a plurality of transfixation wires secured into the lower portion of the leg and at least one foot bone;
a single support rod connected to said frame effective to support said transfixation wires; and
a plurality of adjustable clamps connecting said frame to said support rod and said transfixation wires to said support rod.

2. The apparatus of claim 1 wherein said transfixation wires include a first and a second pair of said transfixation wires.

3. The apparatus of claim 2 wherein said first pair of said transfixation wires is inserted into the medial face of the distal tibia.

4. The apparatus of claim 3 wherein said transfixation wires include a second pair of said transfixation wires that are secured to at least one foot bone.

5. The apparatus of claim 4 wherein the first transfixation wire of said second pair of transfixation wires is inserted into the first metatarsal and the second transfixation wire of said second pair of transfixation wires is inserted into the first cuneiform.

6. The apparatus of claim 1 wherein said support rod is oriented along the anterior-medial aspect of the distal tibia and foot.

7. The apparatus of claim 1 wherein said support frame is configured as a triangle having its base adapted for placement on the surface.

8. The apparatus of claim 7 wherein said support frame includes at least one additional member such that said support frame is configured in a generally rectangular shape.

9. The apparatus of claim 1 wherein a lower portion of said support frame is rounded thereby permitting at least limited rotation of the leg.

10. An external fixation apparatus for supporting a human leg above a surface comprising:
a support frame adapted for placement on the surface;
a plurality of transfixation wires secured into the lower portion of the leg and first metatarsal and the first cuneiform of the foot;
a support rod connected to said frame effective to support said transfixation wires; and
a plurality of adjustable clamps connecting said frame to said support rod and said transfixation wires to said support rod.
